(19) United States
(12) Patent Application Publication
(10) Pub. No.: US 2025/0003878 A1
(43) Pub. Date: Jan. 2, 2025

(54) VALIDATION OF DEGREE OF CURE WITH UV RADIATION BY MEANS OF FLUORESCING WITH EXPOSURE TO UV LIGHT

(71) Applicant: Sunrez Corporation, El Cajon, CA (US)

(72) Inventors: Bret Tollgaard, El Cajon, CA (US); Stuart Mayberry, El Cajon, CA (US)

(21) Appl. No.: 18/634,811

(22) Filed: Apr. 12, 2024

Related U.S. Application Data
(60) Provisional application No. 63/458,837, filed on Apr. 12, 2023.

(51) Int. Cl. G01N 21/64; G01N 33/44

(52) U.S. Cl. CPC G01N 21/6447; G01N 33/442

(57) ABSTRACT

A resin curable by ultraviolet light (UV). More particularly, a resin that changes color during curing to provide confirmation of the completion of curing. The curable resin changes color from the blue of an LED light to an orange while LED light is on the resin and the orange color goes away when LED light is removed.

### RELATED APPLICATION

This application claims the benefit of priority from U.S. Provisional Patent Application No. 63/458,837 filed Apr. 12, 2023.

### BACKGROUND OF THE INVENTION

The present invention is in the technical field of resin curable by ultraviolet light (UV). More particularly, the present invention is in the technical field of resin that changes color during curing. More particularly still, the present invention is in the technical field of resin which changes color during curing to provide confirmation of the completion of curing.

### SUMMARY OF THE INVENTION

The present invention provides a new means of nondestructive testing by which the user of UV curable resin may visually confirm the point at which resin has cured. This greatly enhances quality control as visual inspections are easy to perform. Typical fiberglass repairs will have a hardness test performed on the outside surface to validate the hardness of the laminate and therefore how cured. However, measuring hardness does not convey the level of curing of the laminate underneath the top surface.

The present invention glows bright orange as an indication that the resin is fully cured when exposed to UVA LED 365 nm-405 nm. Typically, while a UVA LED is on and UV cure resin is exposed to it, it will begin to cure but it will maintain the blue color of the LED light whether it's been cured or not. This new resin will change from the blue of the LED light to an orange while the LED light is on. The fluorescent orange color goes away when the LED light is removed.

### BRIEF DESCRIPTION OF THE DRAWINGS

FIGS. 1a and 1b shows samples of typical resin and resin of the invention under ambient light;

FIGS. 2a and 2b shows the samples from FIGS. 1a and 1b exposed to UVA LED light;

FIG. 3a-3d shows a series of samples of typical resin mixed with the present invention under UV LED ambient light; and

FIG. 4a-4d shows the samples from FIG. 3a-3dexposed to UVA LED light.

### DETAILED DESCRIPTION OF THE INVENTION

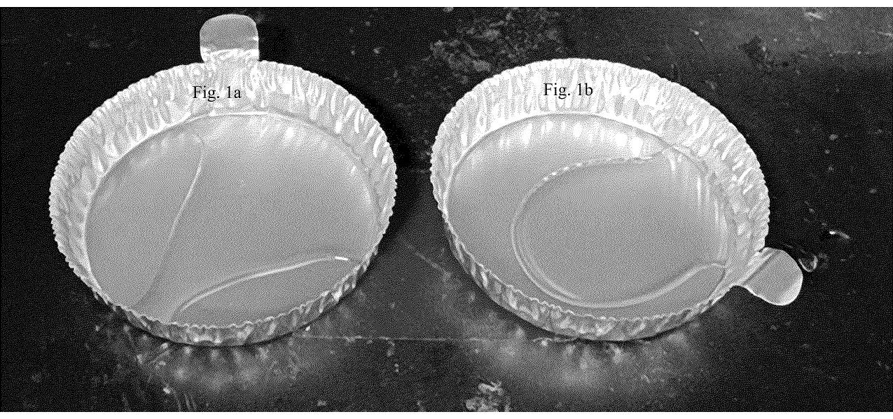

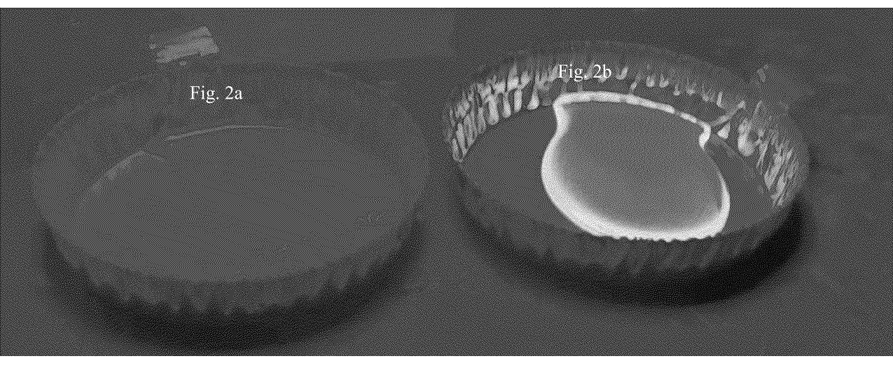

Referring now to the invention in more detail, FIGS. 1a and 1b depict samples of typical resin and resin of the invention under ambient light. FIG. 1a is a typical resin after the curing process while FIG. 1b is the resin of the present invention after the curing process. FIGS. 1a and 1b show the resin samples under normal light. FIGS. 2a and 2b depict the resin samples of FIGS. 1a and 1b exposed to a UVA LED of 365-405 nm wavelength. As is demonstrated in the figures, the typical resin on the left retains the blue color of the LED while the resin of the present invention on the right glows bright orange. As the resin proceeds through the curing process, the amount of cross-linking increases, and as a result, exposure to the LED light results in an increase in fluorescence orange color from the beginning through to the end of the process.

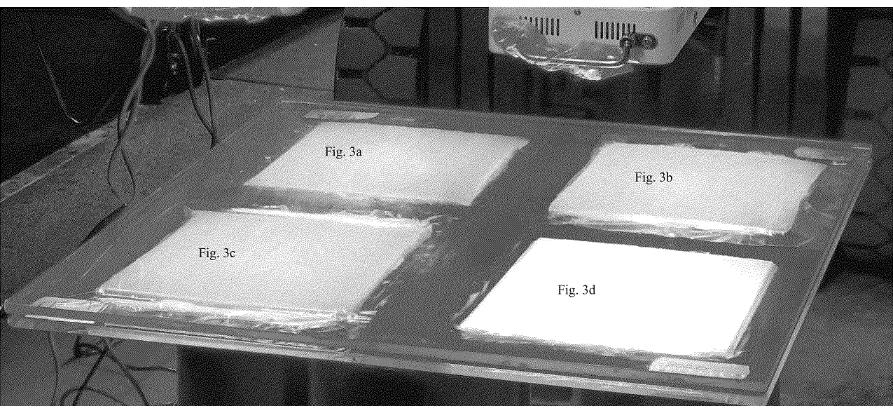

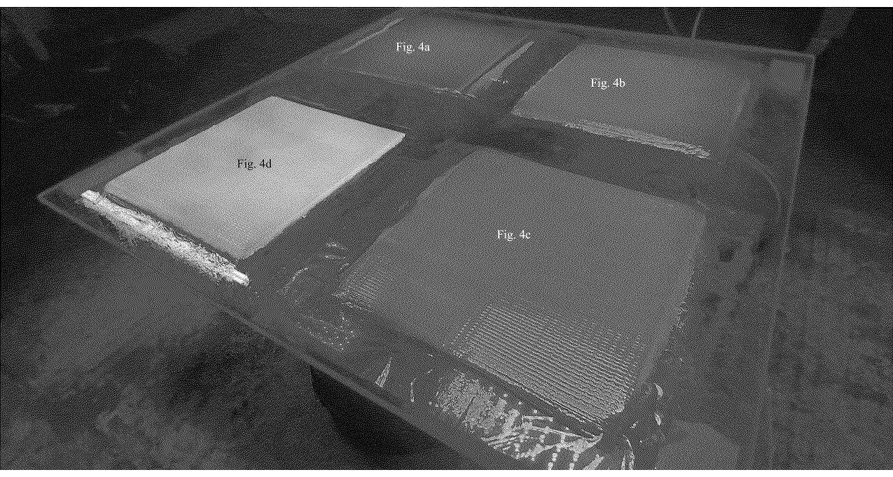

Referring now to FIG. 3a-3d, depicted is a group of cured resin squares. Three traditional resins (FIG. 3a-3c) and one fluorescing resin (FIG. 3d) are shown exposed to UVA LED of 365-405 nm wavelength. FIG. 4a-4d also depict three traditional resins (FIG. 4a-4c) and one fluorescing resin (FIG. 4d) are shown exposed to UVA LED of 365-405 nm wavelength. It is clearly shown in the figures, the typical resin on the left retains the blue color of the LED while the resin of the present invention on the right glows bright orange.

The advantages of the present invention include, without limitation, a means of nondestructive testing of cured resin by providing a visible indicator for confirmation of completion of the curing process.

One embodiment of the present invention provides a curable resin having a bright orange fluorescence when exposed to UVA LED wavelengths in the range of 365 nm to 405 nm.

Another embodiment of the present invention provides a curable resin for use in a variety of industries including, but not limited to, cured-in-place pipes, fiber reinforced repair patches, bathtub and hot tub repair, and the like.

Another embodiment of the present invention provides a curable resin for enhancing quality control through visual inspection of the completion of the curing process.

Still another embodiment of the present invention provides a curable resin that changes throughout the curing process as a function of the amount of cross-linking occurring in the resin.

In a broad embodiment, the present invention provides validation of the degree of cure of a resin by visible fluorescence.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

1. A curable resin having a visible indicator for confirmation of completion of the curing process.

2. The curable resin of claim 1, wherein the visible indicator is comprised of the change in color of the resin following curing.

3. The curable resin of claim 2, wherein the visible indicator is activated by exposure to UVA LED of 365-405 nm wavelength.

4. The curable resin of claim 3, wherein the resin changes color from blue to orange following curing.
